(19) United States
(12) Patent Application Publication
(10) Pub. No.: US 2011/0033499 A1
(43) Pub. Date: Feb. 10, 2011

(54) ANTI-VIRAL NUTRACEUTICAL

(75) Inventor: Adrian Cuthbertson, Hobart (AU)

Correspondence Address: HOGAN LOVELLS US LLP;IP GROUP, COLUMBIA SQUARE, 555 THIRTEENTH STREET, N.W., WASHINGTON, DC, 20004, US

(73) Assignee: MARINE BIOTECHNOLOGY AUSTRALIA PTY LTD, HOBART (AU)

(21) Appl. No.: 12/988,744

(22) PCT Filed: Apr. 21, 2009
(86) PCT No.: PCT/AU09/00487

§ 371 Date: Oct. 20, 2010

(30) Foreign Application Priority Data
Apr. 21, 2008 (AU) 2008901953

(51) Int. Cl. A61K 39/21; A61K 38/17; A61K 39/12; A61K 39/245; A61K 39/25; A61K 39/145; A61P 37/04; A61P 31/12; A61P 31/16; A61P 31/22; A61P 31/18; A61P 31/20

(52) U.S. Cl. 424/208.1; 514/3.7; 514/3.8; 514/4.2; 424/204.1; 424/231.1; 424/230.1; 424/229.1; 424/209.1

(57) ABSTRACT

A method for the prophylaxis or treatment of a viral infection in a mammal is described. The method comprises administering to the mammal an effective amount of a mollusc hemocyanin and/or an active fragment thereof. The hemocyanin may be an abalone hemocyanin.

### FIELD OF THE INVENTION

The invention relates to the use of a mollusc haemocyanin and anti-viral fragments thereof for the prophylaxis or treatment of a viral infection. In another form, the invention provides an anti-viral nutraceutical. The invention finds particular application, but not exclusively, in the prophylaxis or treatment of cold sore outbreaks resulting from Herpes Simplex virus infection.

### BACKGROUND OF THE INVENTION

Abalone is a marine food source which has been used as traditional medicine, including for the treatment of optic atrophy, cataracts and diseases of the lungs. Abalone and other “blue-blooded” arthropods and molluscs do not have the red iron-containing protein haemoglobin as the oxygen carrier in their haemolymph. Instead they utilise a blue copper-containing protein known as “hemocyanin”. Hemocyanins, unlike haemoglobin, are not confined to cells (erythrocytes) but are freely dissolved in the haemolymph.

Mulluscan hemocyanins are very large proteins (approx. 4 MDa), consisting of decamers, didecamers or multidecamers of a 350 or 450 kDa polypeptide subunit. Association of the globular subunits requires either divalent magnesium or calcium ions and competent monomers. As copper type-3 proteins, the oxygen-binding centre of the protein is closely related to phenoloxidases (tyrosinase/catecholoxidase). Especially in Chelicerates (e.g., spiders and scorpions) that lack a gene for phenoloxidase, hemocyanins function as phenoloxidases and play a role in melanin synthesis and subsequent biological processes, such as sclerotisation, wound healing and primary immune defence.

The hemocyanin of the keyhole limpet, Megathura crenulata (KLH), has previously been used as an immuno-stimulatory protein. Originally, KLH was used as an adjuvant carrier protein for immunisation in conjunction with small molecule drugs, e.g., organic compounds or peptides (haptens), which alone are too small to elicit an immune response. Such haptens are recognised by the immune system and antibodies are produced against both the KLH and the haptens. KLH has also been used as a non-specific immune modulator, particularly for immunotherapy in the treatment of bladder cancer.

Previous reports have indicated that abalone juice consisting of exudate from abalone meat has antimicrobial and antiviral activity (Li., 1960; Li, 1962a). When stored for a number of days at 4° C., the juice separates into two layers, the upper layer being brownish yellow in colour and relatively clear while the lower layer is greyish white and cloudy. The upper layer was also reported as occasionally becoming a gelatinous solid.

Fractions of abalone designated as paolin I and paolin II have also been investigated for antimicrobial (paolin I) or anti-viral (paolin II) activity. (Li et al, 1962b; Li et al, 1965; Prescott, 1964); Che, 1991; Der Marderosian, 1969). Paolin is a Chinese term meaning abalone extract. The paolin fractions are prepared by homogenising abalone meat in a blender. They are described as having molecular weights of about 10 kDa or less and were thought to be glycoproteins or mucoproteins. However, this is in apparent conflict with the reported finding that the fractions were thermostable (95° C. for 45 minutes) and not digested by pepsin. Subsequent fractionation indicated the active or actives in the paolin fractions may have a molecular weight of 700 Da or below and be bound to a carrier, which could possibly be glycoprotein or mucoprotein in nature (Li et al, 1965).

Another fraction, designated water-soluble Fraction C, that can be obtained from both abalone and oyster meat has also been described (Li et al, 1962b; Der Marderosian, 1969). Fraction C is prepared by acidification, dialysis and lyophylisation of homogenised meat from these animals, and is provided in the form of a white powder that was also found to be thermostable (95° C. for 45 minutes) and not digested by pepsin. However, the active component(s) in paolin I, paolin II and Fraction C have not been isolated or identified.

Acellular and cellular oyster haemolymph fractions have been reported as having anti-viral activity in vitro as has a crude preparation of dried abalone haemolymph prepared from dialysed “bluish-white” haemolymph collected in a container holding a freely bleeding abalone detached from its shell (Olicard et al, 2005; Carriel-Gomes, 2006; Li et al, 1962b). However, acellular hemolymph at a low protein concentration did not inhibit viral reproduction in a Vero cell/HSV-1 assay system. Moreover, after 48 hours of treatment at increased haemolymph protein concentrations, cell cytotoxicity increased to a high level (80%) and destruction of the Vero cell monolayer was observed with haemolymph protein concentrations above 750 μg/ml, resulting in a low selectivity index (SI). In contrast, in the same study, acyclovir [9-(2-hydroxyethoxymethyl) guanosine at a conc. of 1 μg/ml was found to provide high level protection against cell infection with a low percentage of cell destruction (Olicard et al, 2005).

Hemocyanin polypeptides having an apparent molecular weight of 370 kDa from the abalone Haliotis tuberculata and identified as HtH1 and HtH2 are described in United States Patent Application No. 11/512,044 along with other hemocyanin polypeptide sequences. This application relates to the provision of recombinant hemocyanin protein and asserts that a pharmaceutical composition comprising hemocyanin polypeptide as described is suitable as an anti-parasitic, anti-viral or anti-tumour composition due to either non-specific immunostimulation by the hemocyanin polypeptide or as a result of a specific immune reaction to hemocyanin antigen. The application further asserts such compositions can treat high blood pressure, although no evidence of the described hemocyanin fragments having any of these activities is provided.

Polypeptides with molecular weights of 73 kDa and 75 kDa identified as fragments of hemocyamin and reported to have anti-viral activity have been isolated from penaeid shrimp (Zhang et al, 2004). The polypeptides are described as binding to the shrimp white spot syndrome virus (WSSV) and the fish iridovirus, Singapore grouper iridovirus (SGIV), and to inhibit replication of the fish virus. Three further polypeptides with a molecular weight ranging from 2.7 kDa to 8.3 k Da that are described as having anti-microbial activity and identity with a C-terminal sequence of penaed shrimp hemocyamin have also been reported (Destoumieux-Garzon et al, 2001). However, testing of one of these C-terminal hemocyanin fragments (HCTF) against Herpes Simplex virus type 1 (HSV-1) showed it to not be effective (Destoumieux-Garzon et al, 2001; Carriel-Gomes, 2007).

Human infection by Herpes Simplex virus remains a major health problem in both Westernised and developing countries, and there is an ongoing need for new treatments for these and other virus.

### SUMMARY OF THE INVENTION

In an aspect of the invention there is provided a method for the prophylaxis or treatment of a viral infection in a mammal, comprising administering to the mammal an effective amount of a mollusc hemocyanin and/or an active fragment thereof.

The hemocyanin can be in haemolymph collected from the mollusc or be administered in a purified form. By “purified form” is meant the hemocyanin is at least partially purified. Recombinant hemocyanin or active fragments thereof can also be utilised in methods embodied by the invention.

In another aspect of the invention there is provided the use of a mollusc hemocyanin and/or an active fragment thereof in the manufacture of a medicament for prophylaxis or treatment of a viral infection in a mammal.

In still another aspect of the invention there is provided the use of a mollusc hemocyanin or an active fragment thereof for prophylaxis or treatment of a viral infection in a mammal.

The hemocyanin or active fragment thereof may also be used as an adjuvant for stimulating an immune response to an antigen.

By “active fragment” is meant a fragment of hemocyanin that stimulates an immune response against the antigen or otherwise activates the immune system of the recipient for generation of an immune response against the antigen or virus. The term is also to be taken to encompass fragments that may not stimulate the immune system against a target virus, but can nevertheless inhibit infection and/or replication of the virus (e.g., by binding to the virus) or are otherwise virucidal. In at least some embodiments of the invention, the mollusc will be an abalone.

Accordingly, in another aspect of the invention there is provided a pharmaceutical composition comprising an antigen together with an adjuvant consisting of abalone hemocyanin and/or an active fragment thereof. In this embodiment, the abalone haemocyanin or active fragment thereof can be coupled to the antigen or be simply provided together with the antigen.

The individual treated by a method embodied by the invention can, for instance, be a member of the bovine, porcine, ovine or equine families, a laboratory test animal such as a mouse, rabbit, guinea pig, a cat or dog, or a primate or human being. Typically, the mammal will be a human being.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like that has been included in this specification is solely for the purpose of providing a context for the present invention. It is not to be taken as an admission that any or all of these matters form part of the prior art base or were common general knowledge in the field relevant to the present invention as it existed anywhere before the priority date of this application.

The features and advantages of methods of the present invention will become further apparent from the following detailed description of non-limiting embodiments.

### DETAILED DESCRIPTION OF EMBODIMENTS OF THE INVENTION

As will be understood, haemolymph containing the hemocyanin can be administered in accordance with an embodiment of the invention. The haemolymph can be neat or diluted. By “neat” is meant haemolymph in the form collected from the mollusc. Purified or partially purified hemocyanin can also be formulated into a pharmaceutical composition comprising the hemocyanin together with a pharmaceutically acceptable carrier. Suitable pharmaceutical compositions include pharmaceutical solutions and sterile powders. Such solutions will typically be prepared by incorporating the hemocyanin in the selected carrier. Vacuum drying and freeze-drying techniques can be used to yield a powder of the hemocyanin and any additional desired ingredient from a solution thereof. Solutions (including haemolymph) containing the hemocyanin can be filtered to render the solution sterile.

Active fragments of hemocyanin can be provided by subjecting the hemocyanin to one or more of proteolytic cleavage (e.g., by pepsin and/or trypsin or other protease), treatment with chelating agents, disrupting bonds by chemical treatments, and/or for instance, by sonication.

The purification of hemocyanin and active fragments thereof can be achieved by ammonium sulphate precipitation and/or ion exchange chromatograpy, ultrafiltration and diafiltration techniques well known to the skilled addressee. A method for the purification of molluscan hemocyanin is for example described in US 10/548,609 (US 2006/0160212). A further suitable purification method is described in European Patent No. 1608678. Briefly, in this method, solid material is removed from the haemolymph by centrifugation at 12000g, and the supernatant is passed through a suitably sized column (e.g., Pharmacia Index 140-500) packed with Bio-Rad Macro-Prep High S resin equilibrated with 18 mM acetic acid, 1 mM MgCl₂ and 1 mM CaCl₂ at pH 5.5. The hemocyanin is eluted from the column with 18 mM acetic acid, 1M NaCl, 1 mM MgCl₂ and 1 mM CaCl₂ at pH 5.5. The supernatant is then subjected to buffer exchange by ultrafiltration, before subjecting the supernatant to diafiltration and further purification steps as described.

The hemocyanin will typically have a molecular weight of 4 MDa or greater and more usually, about 5 MDa, 6 MDa, 7 MDa, 8 MDA or greater.

As a further example for the purification of hemocyanin, abalone hemolymph sera may be obtained and subjected to ammonium sulphate precipitation before prefiltration in suitable buffer (e.g, phosphate buffer). Filtration and then ultrafiltration can then be carried out to further purify the hemocyanin, e.g., by use of an ultrafiltration membrane having a suitable molecular weight cut-off to remove smaller proteins but retain the abalone hemocyanin. The appropriate molecular weight cut-off can be determined by analysis of eluted and retained material and employing SDS-PAGE to identify the location of hemocyanin bands. The retained filtrate can then be diafiltered to obtain the abalone hemocyanin in a pharmaceutically acceptable buffer for administration.

Active fragments suitable for use as adjuvants or for the prophylaxis or treatment of viral infections (also referred to herein as anti-viral fragments) in accordance with embodiments of the invention may be selected from the group consisting of a decamer, multi-decamer, and polypeptide subunits of the hemocyanin, and fragments of such decamers and polypeptides. Typically, an active fragment as described herein will have a molecular weight of about 200 kDa, 250 kDa, 300 kDa, 350 kDa or greater. More usually, an active fragment will have a molecular weight of about 400 kDa or greater and most usually, a molecular weight of at least about 750 kDA, 800 kDa or 1 MDa or greater. Suitable hemocyanin fragments and fractions for use in an embodiment of the invention can be identified by T-cell proliferation assays (e.g., cell counts, ³H-thymidine uptake and MTT assays), and any antiviral assays based on cell viability deemed suitable. For example, a cell line infected with the virus of interest or which is exposed to the virus can be treated with haemocyanin and/or active fragment thereof, and the efficacy of the treatment evaluated. For evaluation of anti-HSV activity, African green monkey kidney fibroblast Vero cells (ATCC CCL-81) infected with HSV can be employed (Berġe et al, 1999). Anti-viral fragments of haemocyanin used in methods and compositions embodied by the invention will typically essentially retain the anti-viral activity of the intact hemocyanin. As will be understood, the anti-viral activity of the hemocyanin may be specific against a particular virus or a group of related viruses.

Most usually, however, whole hemocyanin will be administered in accordance embodiments of the invention. As will be also understood, the hemocyanin or active fragment thereof can be a recombinant protein.

The hemocyanin may be administered for prophylaxis or treatment of infection, physical manifestations and/or symptoms by a virus selected from, but not limited to, the group consisting of Herpes viruses and in particular Herpes Simplex viruses (e.g., HSV-1 (predominantly oral) and HSV-2 (predominantly genital)), Herpes Zoster (VZV), Equine Herpesvirus-1 (EHV-1), Feline Herpesvirus-1 (FHV-1), Epstein-Ban virus (EBV), Human Immune Deficiency virus (HIV), Cytomegalovirus (CMV), human papilloma virus (HPV), rhinovirus, influenza virus, and common cold viruses.

The administration of hemocyanin and/or anti-viral fragments thereof as described herein finds particular application in the prophylaxis and/or treatment of cold sores and blisters resulting from HSV-1 or HSV-2 infection. However, the treatment may also find application in limiting transmission of HSV-1, HSV-2 and other viruses such as HIV from infected individuals, and decreasing the risk of transmission of HSV-2 or other viruses from the mother to embryos or newborns. Maternal transmission of HSV-2 to the foetus or newborn can for example lead to encephalitis and death of embryos and babies. A common problem with synthetic drugs is the concern that they might be teratogenic to developing embryos.

Other applications of hemocyanin as described herein may include veterinary uses such as treatment of feline and equine herpes infection symptoms. Further veterinary viruses for which hemocyanin may be used as a therapy (prophylaxis or treatment) include Ceropithecus virus-1 also known as Herpesvirus simiae or herpesvirus B, Simian varicella virus, Equine abortion virus (EHV-1 and EHV-4), BHV-1 also known as Infectious rhinotracheitis or infectious pustular vulovaginitis, BHV-2 also known as Bovine mammilitis, FHV-1 also known as Feline rhinotracheitis, SHV-1 also known as Aujesky's disease or pseudorabies, and CHV-1 also known as canine herpes.

Human herpes simplex virus (HSV) infection is endemic throughout the world, with 60-95% of the adult population estimated to be infected with at least one strain. A recent Australian study examining the prevalence of infection in Australian adults (mean age 45-50) concluded that the seroprevalence (meaning the presence of detectable virus in the blood serum) of HSV-1 and HSV-2 was 76% and 12% respectively, amongst a stratified random sampling of 11,000 individuals. Prevalence was shown to be statistically different dependent on age, sex, geography and indigenous status. In the United States prevalence is predicted to be lower for HSV-1 but higher for HSV-2. The prevalence is significantly higher in some developing countries. For example, the HSV-2 infection rate is greater than 50% in some African countries.

HSV is a member of the herpesviridae family of viruses whose genomes consist of a single large double-stranded DNA molecule. HSV-1 and HSV-2 (HHV-1 and HHV-2) are closely related. After initial infection, all herpesviridae persist permanently in the infected individual, preferentially in neuronal cells and becoming dormant (latency) throughout much of life. Upon certain challenges to the immune system, such as illness, high UV radiation, emotional stress, menses, immuno-impairment or deficiency, the viruses are induced and replicate again, causing a new outbreak. The human immune system generates antibodies and cytotoxic T-cells against the virus, but is not able to eradicate it from the body and cannot prevent further outbreaks.

HSV-1 causes predominantly oral (cold sores), but also genital herpes. HSV-2 is responsible for genital herpes but rarely also may cause the oral form. Current therapies do not eradicate HSV. Outbreaks, such as cold sores, will occur regularly but with varying frequency and severity dependent on the individual. A subject typically initially senses a tingling and/or burning sensation at the onset of a cold sore developing. This sensation is generally followed by a “bump” under the skin which quickly develops into a blister then an open sore. As will be understood, one or more methods as described herein find application for prophylaxis and therapeutic treatment of all stages of the cold sore.

HSV may also cause other primary and recurrent infections of mucous membranes, such as gingivostomatitis and keratoconjunctivitis. HSV is the most common cause of corneal blindness in the United States. Neonatal HSV infection and HSV infections of immuno-compromised individuals (encephalitis, visceral HSV, Kaposi varicella-like eruption) are highly dangerous and associated with high morbidity. In neonates, 60-70% of HSV infections are due to HSV-2 and the remainder, HSV-1. Neonates most often acquire the infection in the intrapartum period via viral shedding from the female genital tract. Other routes of transmission include transplacental passage of the virus and contact spread from an infected caregiver in the postpartum period.

In addition to the direct health effects of HSV infection, many studies and meta-analysis of them have indicated that previous genital HSV-2 infection is a risk factor for spread of HIV. The contribution to spread (attributable risk) is proportional to seroprevalence. Thus, in some African countries where HSV-2 seroprevalence is more than 80%, the attributable risk may rise to 40%.

Herpes viruses infect most if not all vertebrates, including wild animals and fish and companion animals such as cats, dogs and horses. The hallmark of the disease is the establishment of a lifelong infection usually with states of latency from which the virus may reactivate from time to time as it does in humans. The herpes strains infecting these animal species are derived from the alpha herpes viruses, and have a very close relationship with the human viruses HSV-1, HSV-2 and VZV (Herpes Zoster).

Herpes infection in companion and domestic species represents a significant welfare need for prevention and control. For example, feline herpesvirus-1 (FHV-1) is the most common viral pathogen of domestic cats worldwide, with up to 97% of cats having serologic evidence of exposure. It causes an upper respiratory tract and ocular disease in cats known as “feline viral rhinotracheitis”, characterised by conjunctivitis, profuse ocular and nasal discharges, and in some cases, severe keratitis and corneal ulceration. In kittens, the infection can generalise resulting in mortality rates of up to 50%. Although there is routine vaccination of cats, FHV-1 disease remains a common problem and protection may neither be life long of efficacious in preventing infection. Acyclovir has been shown to generally have limited efficacy and can cause toxic effects in cats, but is used in a topical 0.5% ophthalmic ointment to some effect.

In horses, Equine herpesvirus-1 (EHV-1) is a major pathogen which, in addition to causing respiratory disease can also result in abortion and/or neurological signs in infected animals. The infection is widespread and follows the familiar pattern of latency which has been detected in neuronal and lymphoid tissue. Control measures are inadequate and, although vaccines are available, they are not fully protective or give protection of short duration and outbreaks of disease still occur.

The mollusc will typically be a gastropod, and may be selected from the group consisting of abalone, limpets including Megathura crenulata , oysters such as Crassostrea rhizophorae and Crassostrea gigas, muscles, sea snails such as Tegula gallins, scallops, clams such as Mercenaria mercenaria, and conch such as the Queen conch Strombus gigas. Typically, the mollusc will be an abalone. Examples of abalone from which the hemocyanin may be collected include, but are not limited to, Haliotis rubra, Haliotis tuberculata, Haliotis australis, Haliotis aquatilis, Haliotis conicopora, Haliotis coccoradiata, Haliotis corrugata, Haliotis cracherodii, Haliotis diversicolor supertexta, Haliotis fulgens, Haliotis gigantea, Haliotis howensis, Haliotis iris, Haliotis laevigata, Halliotis walallensis, Haliotis sorenseni, Haliotis kamschatkana, Haliotis discus and Haliotis midae amongst others.

The blacklip abalone, Haliotis rubra, is an especially large abalone which can grow up to 200 mm in diameter. For comparison, the European abalone Haliotis tuberculata is only 120 mm in diameter. Haemolymph represents approx. 22% of an abalone by weight while hemocyanin constitutes about 1.2% -1.5% of the haemolymph by weight. The annual Tasmanian catch of abalone is approx. 2,200 tonnes.

When used as an adjuvant, the hemocyanin and/or active fragment(s) thereof can be coupled to the antigen against which the immune response is to be directed. The coupling can be achieved by way of a chemical linker moieties, direct coupling by chemical bonds or for example, by charge attraction between the haemocyanin or fragment(s) and the antigen. The antigen can be any molecule to which an immune response can be generated. The antigen can, for example, be a viral, bacterial or other microbial antigen, or an antigen expressed by cancer cells (e.g., bladder, prostate, leukaemic or breast cancer cells). Typically, the antigen will be a viral antigen such as an outer membrane protein or other suitable antigen. The antigen can be a whole molecule or a fragment thereof containing an epitope presented by the intact molecule. The virus may be any of those identified above such as a Herpes Simplex virus (e.g., HSV-1 or HSV-2).

Hemocyanin can be administered orally, topically including by spraying, transdermally, or by any other suitable route. For oral administration, the hemocyanin can be formulated with an inert diluent or an assimilable edible carrier, and/or can be enclosed in a hard or soft shell gelatin capsule. Moreover, the hemocyanin can be provided in the form of ingestable tablets, buccal tablets, troches, capsules, elixirs, suspensions or syrups. For topical application the mollusc hemocyanin can be formulated into any topically acceptable preparations including creams, lotions, gels and ointments. Such topically acceptable compositions can be applied directly to the site of treatment (e.g., to the cold sore or the site of “tingling” or “burning”).

Pharmaceutical compositions (eg., including so called nutraceutical compositions) containing the hemocyanin can for instance, also incorporate one or more preservatives. In addition, prolonged absorption of the composition may be brought about by the inclusion of agents for delaying absorption such as aluminium monosterate. Tablets, troches, pills, capsules and like can also contain one or more of the following: a binder such as gum tragacanth, acacia, corn starch or gelatine; a disintegrating agent such as corn starch, potato starch or alginic acid; a lubricant such as magnesium stearate; a sweetening agent such as sucrose, lactose or saccharin; and a flavouring agent.

Pharmaceutically acceptable carriers include any suitable conventionally known physiologically acceptable solvents, dispersion media, isotonic preparations and solutions such as physiological saline. Use of such ingredients and media for pharmaceutically active substances is well known. Except insofar as any conventional media or agent is incompatible with the hemocyanin, use thereof is expressly encompassed. It is particularly preferred to formulate parenteral compositions in dosage unit form for ease of administration and uniformity of dosage. Dosage unit form as used herein is to be taken to mean physically discrete units, each containing a predetermined quantity of the hemocyanin calculated to produce a therapeutic or prophylactic effect. When the dosage unit form is a capsule, it can contain the active in a liquid carrier. Various other ingredients may be present as coatings or to otherwise modify the physical form of the dosage unit. For instance, tablets, pills or capsules may be coated with shellac, sugars or both.

The amount of the hemocyanin or active fragments thereof in the composition will be such that a suitable effective dosage will be delivered to the individual taking into account the proposed mode of administration. Typically, a dosage of a composition embodied by the invention will comprise from about 0.5 mg and 50 mg of hemocyanin and/or active fragment thereof, more usually from about 1 mg to about 25 mg most usually from about 1 mg to about 10 mg.

The dosage of the hemocyanin will also depend on a number of factors including whether the active is to be administered for prophylactic or therapeutic use, the viral infection for which the hemocyanin is intended to be administered, the severity of the condition, the age of the individual, and related factors including weight and general health of the individual as may be determined in accordance with accepted medical principles. For instance, a low dosage may initially be given which is subsequently increased at each administration following evaluation of the individual's response. Similarly, frequency of administration can be determined in the same way that is, by continuously monitoring the individual's response between each dosage and if necessary, increasing the frequency of administration or alternatively, reducing the frequency of administration.

Suitable pharmaceutically acceptable carriers and formulations useful in compositions of the present invention may for instance be found in handbooks and texts well known to the skilled addressee, such as “Remington: The Science and Practice of Pharmacy (Mack Publishing Co., 1995)”, the contents of which is incorporated herein in its entirety by reference. The invention is described herein after with reference to non-limiting Examples.

### EXAMPLE 1

### Extraction of Haemolymph from the Blacklip Abalone Haliotis rubra

### 1.1 Selection of Abalone

Abalone were collected from the waters off Tasmania, Australia. The abalone were selected on the basis of appropriate size and fitness, and were free of marks, scars and outward signs of stress and disease. Suitable candidates were conditioned for a minimum period of 5 days in a re- circulating salt water system that was certified free of disease. The temperature of the water was stabilized at 14° C., and the water had a salinity of between 32 and 33 parts per thousand.

1.2 Preparation of Animals for Sera Extraction

Conditioned abalone were drenched with 0.2 μm filtered salt water (Sartorius Sarto™ “P” sterile capsule) to reduce the risk of bacterial contamination of the sera during the extraction procedure. The abalone were then left for a short period inclined at an angle of 30°, foot side up to allow excess rinse water to drain from the body of the animal. The abalone were then wiped dry with commercially available sterile wipes.

### 1.3 Extraction Procedure

Prepared abalone were placed foot side up at an angle of 45° head downwards on a rack such that the abalone were located 400 mm above the extraction table surface. A Terumo Surflo I.V. catheter (14 g×1.73 id×51 mm) was inserted in the base of the neck at an angle of about 40° to the long axis and perpendicular to the plane of the foot of the abalone, until the inner shell was intercepted. The internal needle of the catheter was removed leaving the soft outer plastic catheter in place, and the outer catheter was then gradually withdrawn until the cephalic arterial sinus was intersected. At this point, a flow of sera is observed with a small quantity (5-10 ml) allowed to drain to waste to flush the catheter of any debris collected during the insertion stage. A sterile length (250 mm) of 5 mm silicon tubing is then attached to the outer end of the catheter with the free end of the tubing directed into a 1 litre sterile Nalgene bottle. Up to 150 ml of sera can be drained from individual abalone using this method without causing mortality but this is dependent on the weight of each animal. The process is repeated until each 1 litre Nalgene bottle has been filled and the required amount of sera has been extracted. The Nalgene bottles were then sealed and stored at below 5° C. until use.

### 1.4 Filtration

Using a Watson Marlow 520 transfer pump, the collected sera was pumped through a Millipore “Polysep Opticap” XL 4 filter (capsule No. 0045) with a nominal pore size of 1.0/0.5 μm. This filter was connected via sterile silicon tubing to a Millipore “Opticap” filter with a “Durapore” 0.22 μm nominal pore size. (Cat. No. KVGLS4HH3). Delivery to a “Flexboy” 10 litre “Stedim” bag and associated (attached) 150 ml “Stedim” sampling bag was by way of an attached “Flexboy sterile Transfer Set” with ⅜″ fittings.

The weight and date of manufacture of each bag of sera filtered to 0.22 μm is recorded and the product stored under refrigeration.

### EXAMPLE 2

### Characterisation of Abalone Hemocyanin

### 2.1 Preparation of Hemolymph Samples

Hemolymph from the Australian blacklip abalone (Haliotis rubra) was freshly extracted and subjected to centrifugation at 18000 g for 20 minutes to remove debris in the crude sera. The supernatant was then passed through a series of filters with decreasing pore size, i.e., 6 μm, 2.7 μm, 1.2 μm, 0.45 μm and 0.22 p.m. The sera was effectively sterilised by passage through the 0.22 μm filter.

### 2.2 Particle Size and Size Distribution in Abalone Sera

Particle size and size distribution of the hemocyanin was measured by Zetasizer Nano-ZS (Malvern Instruments, UK) based on the Brownian motion of molecules using dynamic light scattering (DLS, QELS). A disposable sizing cuvette was filled with the hemocyanin sample and placed in the measurement chamber of the Nanosizer. Size and size distribution measurements were repeated in triplicate. The size of fresh abalone hemocyanin (AH) was determined to be 45.7 nm and the size distribution polydispersity index (PDI) was only 0.059, which is relatively monodisperse. Notably, heat and/or pH can change the size dramatically, but the abalone hemocyanin remains at approximately 45.7 nm when stored at low temperature (e.g., 2-8° C.) or when subjected to centrifugation.

### 2.3 Molecular Weight of Abalone Hemocyanin

The molecular weight of the hemocyanin was measured by Zetasizer Nano-ZS (Malvern Instruments, UK) employing light scattering (SLS). The sera samples were diluted with Milli-Q water to 5 concentration levels giving hemocyanin concentrations ranging from approximately 0.05 mg/mL to 0.3 mg/mL. The value of the differential refractive index increment do/dc for hemocyanin is 0.194 cm³/g, and toluene was used as the reference. Optically matched quartz cells were used for each sample, i.e. toluene, base solvent and hemocyanin at different concentrations. The molecular weight of hemocyanin was calculated by the software employed to be 8180 kDa. Notably, high temperatures caused hemocyanin aggregation, while low or high pH (e.g. below pH 5.0 or above pH 9.0) tends to degrade the hemocyanin into fragments.

### 2.4 Solubility of Abalone Hemocyanin

Hemolymph samples were obtained as described under Example 2.1 above and pelleted using a Beckman TL 100 Ultracentrifuge at 100,000 rpm for 1 hour at 4° C., resulting in a clear supernatant and a dark blue sediment. The sediment was covered by Parafilm and stored at 4-8° C. overnight. The sediment was relatively insoluble and did not totally re-dissolve even with 4 hours of vortexing. The particle size of the resuspended hemocyanin agglomeration was measured by QELS (as per Example 2.4 above) which indicated an agglomerated particle size of 18,813 nm.

### 2.5 Melting Point of Hemocyanin and pH Related Denaturation

The melting point of hemocyanin and pH related denaturation was measured by differential scanning calorimetry (DSC). The hemocyanin fraction was separated from the sterile filtered sera by ultracentrifugation (543,000 g for 1 hr at 4° C.). The pellet/sediment was then immediately resuspended with 20% sucrose solution (w/w, sterile filtered) to the original volume prior to the ultracentrifugation. 1 mL hemocyanin was then added to 3 mL of buffer and left for 2 hours. The buffers used were acetate buffer pH 4.0, 5.0 and phosphorate buffer pH 9.0, 10.0, 11.0. Approximately 10-20mg of sample was placed in the centre of an aluminium pan, and then sealed with an aluminium inverted cover by the DSC sample press. DSC measurements were performed using a Q20 model (TA Instrument—Waters LLC, New Castle, Del., USA). A heating rate of 5 ° C. /min in the 25-180° C. temperature range was chosen. The melting point of abalone hemocyanin was determined as 77.9° C. Denaturation of the hemocyanin was noted below pH 5.0 and above pH 9.0.

### 2.6 Hemocyanin Copper Content and Hemocyanin Concentration in Serum

Serum copper content was determined using an atomic absorption spectrometer (Model AA140, Varian) at 324.8 nm. Hemocyanin samples of 0.4 mL were mixed with 1.2 mL HNO₃ (65%) and added into a clear vial with a PTFE liner screw cap. The yellow mixture was heated in a boiling water bath for 7 hours leaving a clear solution which was left overnight. The solution was transferred to a 10 mL volumetric flask and Milli-Q water was added giving a final HNO₃ concentration of 7.8% v/v. To calibrate the measurements, standard Cu solutions were prepared in 2% HNO₃. One hemocyanin didecamer contains 320 Cu atoms. The copper content in abalone hemolymph was measured as 29.0 μg/mL, which corresponds to an estimated hemocyanin content of 11.7 mg/mL.

### EXAMPLE 3

### Treatment Cold Sore Suffers with Hemocyanin from the Black Lip Abalone Haliotis rubra—Anecdotal Study

A total of 8 human cold sore sufferers were administered 25 ml of whole haemolymph prepared as in Example 1 or 5mg (5×1 mg) of hemocyanin in purified form (AH-CAPS) daily for a period of 10 days. Diaries were kept by the subjects for the duration of the study. Peripheral blood was collected from the subjects prior to the start of the study and immediately following the end of the study period for natural killer cell (NK) and viral induced proliferation assays. The blood assay results are summarised in Example 7

### 3.1 Subjects and Results

### (i) 29662 Female—50-60 yrs

No underlying medical condition. A chronic sufferer with at least 6 cold sore outbreaks per year. The cold sores are induced by stress and also triggered by peanuts.

Following ingestion of whole haemolymph, this subject noticed faster healing and less blistering. The ingestion of peanuts did not trigger cold sore development.

This subject was also subsequently administered AH-CAPS and has noticed that the cold sores do not develop. Rather, at most, a small “bump under the skin” is experienced.

### (ii) 82822 Female—50-60 yrs

No underlying medical condition. An occasional sufferer with 3-4 cold sore outbreaks per year. Cold sores induced are induced by stress. Peanuts and chocolate are also an aggravating factor.

This subject was administered whole haemolymph. No cold sores were experienced during the period of the study, and cold sores have not reappeared since the completion the study. This subject does experience a “pre-tingle” or burning sensation, but this does not development into a cold sore. The subject also ate peanuts and chocolate during the study period.

(iii) Female—50-60 yrs

No underlying medical condition. An occasional sufferer with 3-4 cold sore outbreaks per year. Cold sores are induced by stress.

This subject was administered whole haemolymph and took the extract when experiencing early symptoms of cold sores developing. The cold sores did not develop fully and dried up much more rapidly.

This subject also took 1 mg HA-CAPS the next time the symptoms of a cold sore were experienced Again, the colds sore did not fully develop and at most, a small blister was observed. Approximately 6 months after originally consuming the whole haemolymph, the subject has not developed a cold sore even after returning to work after a holiday period, a time when cold sores normally developed. The subject occasionally experiences a ‘tingle’ sensation which would normally signal the onset of a cold sore, but no cold sores develop.

### (iv) Female—40-50 yrs

No underlying medical condition. An occasional sufferer with 3-4 cold sore outbreaks per year. Experiences multiple cold sores all over her face.

This subject was administered whole haemolymph. Approximately 7 months after the completion of the study, this subject had not experienced any cold sores although she has reported experiencing “tingling sensation” or “bump” under the skin. This subject also reported a longer interval between such episodes.

(v) 45103 male 50-59 yrs

No underlying medical condition and not normally a cold sore sufferer. This subject was a sibling of one of the subjects in the study. Ironically, a cold sore appeared on the tip of his nose which he accidentally wiped into his eye where an extremely painful cold sore developed. The subject was advised by a general practitioner that a 10-14 day healing time would be required.

This subject was administered 5 mg AH-CAPS daily for 1 week and within 2 days the pain had diminished. Healing had occurred within 5 days.

### (vi) 57951 Male 30-39 yrs

No underlying medical conditions. A chronic sufferer with 8 cold sore outbreaks per year with a lengthy healing time (at least 10 days). The subject is in public eye and feels conspicuous when outbreaks occur.

Following administration of AH-CAPS, the subject has experienced a reduced number of cold sore outbreaks and a more rapid healing time (4-5 days rather than 10 days). The cold sores are also now reduced to a bump under skin with much less blistering.

The subject had a small bump on his lip at the end of the study but it did not progress to a blister. Seven months after the study the subject has had occasional “bumping” during that time and has used Zovirax™ cream to treat burn or tingling symptoms, which would then last 3 hours with no bump produced. The subject since noticed more persistent bumping under skin and has developed a cold sore blister but has indicated he is very happy with the hemocyanin treatment.

(vii) 93778 male 30-39 yrs

No underlying medical conditions. A chronic sufferer with 6-8 cold sore outbreaks per year. The cold sores are induced by sunburn, windburn and after working night shifts.

Following treatment with the AH-CAPS, the subject went fishing and also got sunburnt during the study, but did not develop cold sore. This has never been previously experienced by the subject, as this would always result in a cold sore outbreak.

On returning to night shift a cold sore developed (4 days before end of trial), but viral culture negative. This cold sore did not progress to blister, and had reduced severity.

Approximately 7 months after the study, the subject has experienced no further outbreak of cold sores. It would have been expected that a cold sore episode would on average have been experienced every 6 weeks or so, particularly after working a night shift roster.

(viii) 88038 Female 50-59 yrs

No underlying medical condition. Chronic sufferer with about 8 outbreaks per year making her feel that she always has a cold sore. As this subject always works night shift, she feels that she is run down, and gets cold sores “all the time”.

Following the treatment with AH-CAPS a cold sore did develop but it did not blister, with just bump under skin being experienced. Consequently, the severity of the episode was significantly reduced. Viral culture was negative.

### 3.2 Discussion

All of the subjects in this study reported a substantial improvement following taking the hemocyanin with less cold sore episodes and reduced intensity of cold sores. Accordingly, the data shows the efficacy of treatment with hemocyanin for prophylaxis and therapy of HSV/cold sore outbreaks. In particular, a reduction in the frequency of cold sore outbreaks and a decrease in healing time was observed.

### EXAMPLE 4

### Treatment with Haemolymph from the Abalone Haliotis rubra—Study on Chronic Sufferers

Chronic subjects were considered as those that have at least 6 outbreaks of cold sores per year. Subjects were administered a 5 mg (5×1 mg) dose of hemocyanin/placebo (5 capsules) daily for 30 days. Daily saliva samples were collected for 10 days prior to capsule taking and for the last 10 days of capsule taking. Peripheral blood was also collected from subjects prior to the administration of the hemocyanin and immediately after the cessation of capsule taking for natural killer cell (NK) and viral induced proliferation assays.

Peripheral blood samples were also assayed for white cell counts and flow cytometry. Total DNA was extracted from saliva samples using the MagNA Pure LC automated extraction system (Roche) and DNA concentrations calculated for real time PCR. If the concentration of DNA in a sample was not greater than 10 ng/uL, then the DNA was precipitated using ethanol, resuspended in 20 uL and concentration re-calculated.

(NB: #073—Saliva samples were collected for 7 days in both instances (i.e., prior to capsule taking and during the final phase of the study period as outlined above). This subject also only consumed capsules for 21 days due to time constraints).

### 4.1 Subject Diary Entries

“PRE” is the 1st day of treatment; “POST” is the last day of treatment.

| | | | |
|---|---|---|---|
| #009: | Pre 19/02/2007 | Post 29/03/2007 | AH-CAPS |
| #037: | Pre 21/02/2007 | Withdrawn | Placebo |

#047: Day 28: Have very small cold sore at base of nose.
Day 29: Has not got any bigger and seems to be going.
Day 30: Going.
Day 32: Got very small cold sore on right side of face (never had them there before), went through blister stage very quickly.
Day 33: Has not got any worse. Have small red mark that is a little tender.
Day 34: Going.

#082: Day 7: Started feeling cold sore.
Day 8: Still feeling cold sore.
Day 9: Cold sore only cracked lip.
Day 19: Started to feel cold sore on lower lip (near cracked lip).
Day 21: Cold sore clearing already.
Day 33: Thought getting cold sore on nose today.
Day 34: Nose still itchy—maybe cold sore?
Day 35: No cold sore.

#083: Day 17: Tingling on upper lip—refrained from putting Zovirax on it.
Day 18: No cold sore coming out—good.
Day 20: Kept checking lip but no cold sore.

#103: Day 18: Started getting cold sore. Only got one blister, didn't put any cream on it.
Day 21: Blister cleared up a lot quicker than usual.

#118 NB: Didn't write anything in diary, but when questioned at the end of the trial this subject stated that she hadn't had any cold sores over the duration of the trial and that this was unusual as she had been getting a lot of cold sores prior to the trial.

NB: The remaining six subjects (out of the total of 11) reported no change in their cold sore occurrences.

### 4.2 Discussion

The reports from the subjects in this study support the efficacy of treatment with hemocyanin for prophylaxis and therapy of HSV/cold sore outbreaks. In particular, a decrease in the intensity of cold sore outbreaks and in healing time was observed, with some cold sores not progressing to the blister stage.

### EXAMPLE 5

### Treatment of Chronic Cold Sore Sufferers with Haemolymph from Haliotis rubra

These subjects were those that have at least 6 cold sore outbreaks per year, but were not included in the chronic sufferers study described in Example 4. Saliva samples were taken daily for 5 days. Following this, subjects took 25 ml of haemolymph daily (identified as “Extract” in the tables below) for 10 days prior to the collection of daily saliva samples for a further 5 days. Peripheral blood was again collected from subjects before and after completing the course of haemolymph. Peripheral blood mononuclear cells (PBMC) were extracted from the blood and used for cytokine analysis and HSV-1 cell proliferation assays. Total DNA was extracted from saliva samples using the MagNA Pure LC automated extraction system (Roche) and concentrations calculated for real time PCR. Again, if the concentration of DNA was not greater than 10 ng/uL the DNA was precipitated using ethanol, resuspended in 20 uL and concentration re-calculated. A total of 50 ng (10 ng/uL) per sample was used for real time PCR analysis. Daily diaries were kept by all subjects.

### 5.1 Subjects

| | | |
|---|---|---|
| #804: | Pre 11/05/2007 | Post 21/05/2007 |
| #807: | Pre 11/05/2007 | Post 21/05/2007 |
| #840: | Pre 11/05/2007 | Post 21/05/2007 |
| #858: | Pre 11/05/2007 | Post 21/05/2007 |
| #887: | Pre 18/05/2007 | Post 28/05/2007 |
| #915: | Pre 18/05/2007 | Post 28/05/2007 |

### 5.2 Subject Diary Entries

#804: Nothing to report.

#807: Nothing to report.

#840: Got lip pain on day 6 which lasted for 2 days, but did not result in a blister.

#858: Already had a cold sore that had lasted 1 week prior to the trial. This went away within 3 days of starting the trial (would have expected the cold sore to last another week).

#887: Had dry lips on days 4 and 5 which progressed to a bump on days 6 and 7, but did not progress further to a blister.

#915: Got a tingle on days 2 and 3 but did not progress to a blister.

Out of the 6 subjects, 4 indicated treatment with the haemolymph was helpful. Two out of the 6 did not experience any benefit or otherwise, and so did not report anything.

### 5.3 Natural Killer Cell Activity

No significant natural killer cell (NK) activity was found in blood samples from any of the subjects.

### 5.4 Detection of HSV-1

The samples from the 10 day (10D) cold sore study were thought most likely to show the presence of virus (HSV-1) by PCR analysis as the majority of the subjects had active cold sore lesions when saliva samples were taken. However, no viral DNA was detected.

### 5.5 Cyokine Analysis

The data from the PHA induced cytokine analysis was unexpected as the levels of all cytokines assayed decreased upon mitogen (PHA) stimulation. A significant reduction in IL-10 production was observed. The assay was conducted over 72 hours. Analysis over 24-48 hours may have resulted in a different outcome. The results are set out in Table 1. NB: The #840 post sample was contaminated and so was not used.

TABLE 1
PHA-induced cytokine stimulation data

| | | | | | | |
|---|---|---|---|---|---|---|
| 10D HSV | 804 | 807 | 858 | 887 | 915 | T test |

TNF

| | | | | | | |
|---|---|---|---|---|---|---|
| Pre Extract | 4903.569 | 2939.723 | 1867.800 | 1585.877 | 1683.185 | 0.553338514 |
| Post Extract | 1712.800 | 1003.954 | 5805.108 | 590.877 | −122.585 | |

IFN

| | | | | | | |
|---|---|---|---|---|---|---|
| Pre Extract | 3844.202 | 3537.893 | 2792.485 | 4016.757 | 4021.489 | 0.281248147 |
| Post Extract | 3401.615 | 5670.700 | 4030.953 | 3927.167 | 1110.132 | |

IL10

| | | | | | | |
|---|---|---|---|---|---|---|
| Pre Extract | 5241.88 | 7534.22 | 6603.14 | 5352.24 | 4318.91 | 0.008222194 |
| Post Extract | 2033.77 | 2659.45 | 1545.93 | 1816.20 | 3302.24 | |

IL4

| | | | | | | |
|---|---|---|---|---|---|---|
| Pre Extract | −0.945 | 0.349 | −1.435 | −0.306 | −0.357 | 0.572028571 |
| Post Extract | −0.749 | −0.396 | −0.0651 | −1.44 | −1.533 | |

### 5.6 Cell Proliferation Assay

There was no significant difference in cell proliferation between pre and post treatment samples following HSV-1 mitogen stimulation.

### 5.7 Discussion

The reports from the subjects in this study support the efficacy of treatment with hemocyanin for prophylaxis and therapy of HSV/cold sore outbreaks. In particular, a decrease in the intensity of cold sore outbreaks and in healing time was observed, with some cold sores not progressing to the blister stage.

### EXAMPLE 6

### Treatment of Acute Cold Sore Sufferers with Haemocyanin from Haliotis rubra

Acute subjects were considered those that have outbreaks of cold sores on less than 6 occasions per year. For the purpose of this study 2 groups of subjects were identified, namely “Acute A” subjects being those those with less than 6 outbreaks of cold sores a year (e.g., 3-4 or less), and “Acute B” subjects being those with close to 6 outbreaks a year. Acute B subjects were considered likely to have an outbreak of cold sores over the 3 month period study period.

Saliva samples were collected weekly from these subjects over the 12 week period. Peripheral blood was collected prior to starting the trial and immediately after cessation of capsule taking. If an outbreak of cold sores occurred, the subject immediately started taking a 1 mg (1 capsule) dose of AHC or placebo for 10 days. Peripheral blood was collected immediately after taking the last capsule. Daily diaries were kept by all subjects.

Peripheral blood mononuclear cells were extracted from blood and used for natural killer cell activity assays. White cell counts and flow cytometry analysis was also conducted on blood samples. The blood assay results are summarised below in Example 7.

As no viral DNA was detected in saliva from the chronic sufferers in the 10 day study reported in Example 6, the saliva collected in this study was stored frozen and DNA was not extracted from the samples.

### 6.1 Acute A Subjects

| | | | |
|---|---|---|---|
| #195: | Pre 07/03/2007 | Post 09/05/2007 | AH-CAPS |
| #205: | Pre 07/03/2007 | Post 09/05/2007 | AH-CAPS |
| #209: | Pre 07/03/2007 | Post 09/05/2007 | AH-CAPS |
| #271: | Pre 26/03/2007 | Post 26/04/2007 | AH-CAPS |
| #323: | Pre 30/03/2007 | Post 08/06/2007 | AH-CAPS |
| #324: | Pre 26/03/2007 | Post 28/05/2007 | Placebo |

(NB: #271 began taking capsules immediately so the post date is earlier.)

### 6.2 Natural Killer Cell Activity

No significant natural killer cell (NK) activity was found in blood samples from any of the subjects.

### EXAMPLE 7

### Blood and Saliva Sample Results

Results for blood, saliva, and related assays described in Examples 1-6 above are summarised in Tables 2 and 3.

TABLE 2
Immune study on healing volunteers

| | | | | AH-CAPS- | |
|---|---|---|---|---|---|
| | Haemolymph | Haemolymph | AH- | placebo | AH- |
| | (Extract) | (Extract) | CAPS | 5 x 1 | CAPS |
| | 25 ml- | 25 ml- | 5 x 1 mg- | placebo | 1 mg- |
| | 10 days | 30 days | 30 days | 30 days | 30 days |
| Biochemistry | No significant | No significant | No significant | Overall-no | Overall-no |
| | changes | changes | changes | significant | significant |
| | Cholesterol | Cholesterol | Cholesterol | changes | changes |
| | changing in right | changing in right | changing in right | No changes | No changes |
| | direction | direction | direction | on | on |
| | (LDL↓HDL↑) | (LDL↓HDL↑) | (LDL↓HDL↑) | cholesterol | cholesterol |
| Haematology | Overall-no | Overall-no | Overall-no | Overall-no | Overall-no |
| | significant | significant | significant | significant | significant |
| | changes. | changes. | changes. | changes. | changes. |
| | Platelets | Platelets | Clotting time | | |
| | increased | increased | increased. | | |
| | slightly. | slightly. | | | |
| Lymphocyte | Non-significant | Non-significant | Non-significant | Overall-no | Overall-no |
| numbers | increase in | decrease in | decrease in | significant | significant |
| | lymphocyte | lymphocyte | lymphocyte | changes. | changes. |
| | proliferation, but | numbers | numbers | | |
| | only at the lower | | | | |
| | stimulus levels. | | | | |
| Lymphocyte | Minor | Not done | Minor | Overall-no | Overall-no |
| proliferation | significant | | significant | significant | significant |
| | reduction in | | reduction in | changes. | changes. |
| | lymphocyte | | lymphocyte | | |
| | proliferation at | | proliferation at | | |
| | lower stimulus | | higher stimulus | | |
| | levels | | levels and only | | |
| | | | with PWM | | |
| | | | (not PHA) | | |
| NK | Minor but | Not done | Non-significant | Overall-no | Overall-no |
| cytotoxicity | significant | | reduction in NK | significant | significant |
| | increase in NK | | cytotoxicity. | changes. | changes. |
| | cytotoxicity but, | | | | |
| | only at the lower | | | | |
| | stimulus levels. | | | | |

TABLE 3
Cold sore studies

| | | | Acute cold | Chronic cold sore |
|---|---|---|---|---|
| | | Chronic cold sore | sore | Haemolymph |
| | Haemolymph | AH-CAPS | AH-CAPS | (Extract) - |
| | (Extract) | 5 × 1 mg - 30 days | 1 mg - 10 days | 10 days |
| Lymphocyte | Not done | No changes | Insufficient | |
| levels | | observed | data | |
| NK | Not done | Non-significant | Non- | |
| cytotoxicity | | increase in NK | significant | |
| | | cytotoxicity | increase in NK | |
| | | | cytotoxicity | |
| Cytokine | Not done | Not done | Not done | Reduction in all |
| production | | | | cytokine levels: |
| | | | | Significant drop in |
| | | | | IL-10 production. |
| Virus levels - | Not done | Unable to detect | Not done | Unable to detect HSV |
| saliva | | HSV virus DNA in | | virus DNA in any |
| | | any samples (pre or | | samples (pre or post |
| | | post capsules) | | extract) |
| HSV - | Not done | Not done | Not done | No significant |
| proliferation | | | | difference in cell |
| | | | | proliferation |
| Diaries | Strongly suggest that | 5 out of 11 subjects | Not done | 4 out of 6 subjects |
| | extract is reducing cold | provided positive | | provided positive |
| | sore levels if taken | feedback, e.g., cold | | feedback. |
| | early. Number of | sores did not | | |
| | recurrences reduced. | develop, cleared up | | |
| | Summary - In total, 8 | more quickly. | | |
| | of 8 subjects reported | 6 out of 11 subjects | | |
| | substantial | reported no change | | |
| | improvement. | to their cold sore | | |
| | | frequency and/or | | |
| | | healing. | | |

### 6.1 Discussion

Subjects made many positive reports following the taking of purified haemocyanin capsules (AH-CAPS) or neat haemolymph (“Extract”). There were trends found for increasing immunological function in some assays. However, these were often not statistically significant (although they may be biologically significant). No detrimental effect on blood chemistry was observed. Overall, subjects generally reported less frequency in HSV/cold sore outbreaks with less intensity, and reduced healing time.

Although the invention has been described with reference to particular embodiments, it will be appreciated by those skilled in the art that numerous variations and/or modifications may be made departing from the invention as broadly described. The present embodiments are, therefore, to be considered in all respects as illustrative and not restrictive.

### REFERENCES

1. Bergé J. P. et al. “Antiviral and anticoagulant activities of a water-soluble fraction of the marine diatom Haslea ostrearia” (1999), Planta Med. 65, 604-609.
2. Carriel-Gomes et al. “Evaluation of antiviral activity in hemolymph from oysters Crassostrea rhizophorea and Crassostrea gigas” (2006), Aquat. Living Resource. 19:189-193.
3, Carriel-Gomes et al. “In vitro antiviral activity of antimicrobial peptides against herpes simplex virus 1, adenovirus and rotavirus” (2007), Mem. Inst. Oswaldo Cruz, Rio de Janeiro, Vol. 102(4): 469-472, June.
4. Che, C-Tao. “Marine products as a source of antiviral drug leads” (1991), Drug Development Research, 23:201-218.
5. Der Maderosian, A. “Marine Pharmaceuticals” (1969), J. Pharm. Sciences, 58(1).
6. Destoumieux-Garzon et al. “Crustacean Immunity” (2001), J. Biol. Chem, 276 (50):47070-47077.
7. Li, C. P. “Antimicrobial effect of abalone juice” (1960), Proc. Soc. Exp. Biol. Med. 103:522-524.
8. Li et al. “Antiviral activity of a fraction of abalone juice” (1962a), Proc. Soc. Exp. Biol. Med. 109:534-538.
9. Li, C. P. “Antimicrobial agents from molluscs” (1962b), Trans N. Y. Acad. Sci., Series II, 24:504-509.
10. Li et al. “Antiviral activity of paolins from clams”, (1965), Annals. N Y. Acad. Sci, 374-382.
11. Prescott et al. “Isolation and characterization of antiviral substances from marine animals” (1964), Fed. Proc. 23:508.
12. Olicard et al. “Putative antiviral activity in hemolymph from adult Pacific oysters, Crassostrea gigas” (2005), Antiviral Research, 66:147-152.
13. Zhang et al. “Antiviral properties of hemocyanin isolated from shrimp Penaeus monodon” (2004), Antiviral Research, 61:93-99.

1. A method for the prophylaxis or treatment of a viral infection in a mammal, comprising administering to the mammal an effective amount of a mollusc hemocyanin or an active fragment thereof.

2. A method according to claim 1 wherein haemolymph is administered to the mammal.

3. A method according to claim 1 wherein the hemocyanin is administered to the mammal is in at least partially purified form.

4. A method according to claim 1 wherein the hemocyanin or active fragment thereof has a molecular weight of about 4 MDa or greater.

5. A method according to claim 1 wherein the mollusc is an abalone.

6. A method according to claim 5 wherein the abalone is Haliotis rubra.

7. A method according to claim 1 wherein the viral infection is an infection by a virus selected from the group consisting of Herpes Simplex viruses (HSV), Herpes Zoster virus (VZV), Equine Herpesvirus-1 (EHV-1), Feline Herpesvirus-1 (FHV-1), Epstein-Barr virus (EBV), Human Immune Deficiency virus (HIV), Ceropithecus virus-1, EHV-1, EHV-4, BHV-1, BHV-2, FHV-1, SHV-1, CHV-1, CMV, HPV, Rhinovirus, common cold viruses and influenza virus.

8. A method according to claim 7 wherein the virus is a Herpes Simplex virus.

9. A method according to claim 8 wherein the virus is selected from the group consisting of HSV-1 and HSV-2.

10. A pharmaceutical composition comprising an antigen together with an adjuvant consisting of abalone hemocyanin and/or an active fragment of the hemocyanin.

11. A pharmaceutical composition according to claim 10 wherein the antigen is an antigen of a virus.

12. A pharmaceutical composition according to claim 11 wherein the virus is selected from the group consisting of Herpes Simplex viruses, Herpes Zoster virus (VZV), Equine Herpesvirus-1 (EHV-1), Feline Herpesvirus-1 (FHV-1), Epstein-Barr virus (EBV), Human Immune Deficiency virus (HIV), Ceropithecus virus-1, EHV-1, EHV-4, BHV-1, BHV-2, FHV-1, SHV-1, CHV-1, CMV, HPV, Rhinovirus, common cold viruses and influenza virus.

13. A pharmaceutical composition according to claim 12 wherein the virus is a Herpes Simplex virus.

14. A pharmaceutical composition according to claim 13 wherein the virus is selected from the group consisting of HSV-1 and HSV-2.

15-16. (canceled)
